(19) United States
(12) Patent Application Publication
(10) Pub. No.: US 2022/0215937 A1
(43) Pub. Date: Jul. 7, 2022

(54) System and method for adjudicating healthcare trades

(71) Applicant: Arthur Morris Gelber, Ridgefield, CT (US)

(72) Inventor: Arthur Morris Gelber, Ridgefield, CT (US)

(21) Appl. No.: 17/300,667

(22) Filed: Sep. 20, 2021

Related U.S. Application Data
(63) Continuation of application No. 16/501,815, filed on Jun. 11, 2019, now abandoned.
Application No. 16/501,815 is a continuation-in-part of application No. 15/731,087, filed on Apr. 17, 2017, now abandoned.
(60) Provisional application No. 62/323,409, filed on Apr. 15, 2016.

(51) Int. Cl. G16H 40/20; G16H 10/60; G16H 40/40; G06Q 30/08

(52) U.S. Cl. CPC G16H 40/20; G06Q 30/08; G16H 40/40; G16H 10/60

(57) ABSTRACT

System and method for pairing an electronic exchange system with a health care expert system program to mitigate the risk of overpaying for health care and to ensure integrity to financial transactions. During the negotiations of health care, the invention instantly monitors and/or audits financial transaction for compliance, wherein mitigating the risk of overpaying for health care. The system collects knowledge in the form of rules (if-then-form) and store them in any store. An inference engine picks the rules according to the requirements and runs them on input data or query. The system undertakes to always buy or sell health care at specified prices. The system buys and sells health care for its own account, inventory. Additionally, it buys and sells from its inventory. By purchasing health care directly from the exchange, the system can reduce administrative costs by 40%. There is no balance or surprise billing.

### TECHNICAL FIELD

The present invention is directed towards electronic trading. More specifically, the present invention is directed to processes, tools, controls, and function to mitigate excessive costs for health care.

### BACKGROUND

Electronic trading is generally based on a host exchange, one or more computer networks, and client devices. Subscribing traders are connected to an exchange's electronic trading platform by way of communication links to facilitate real time electronic messaging between themselves and the exchanges. The electronic trading platform includes at least one electronic market, which is at the center of the trading system and handles the matching of bids and offers placed by the traders for that market. The electronic messaging includes market information that is distributed from the electronic market to the traders via an electronic data feed. Once the traders receive the market information, it may be displayed to them on their trading screens. Upon reviewing the information traders or users can take certain actions including the actions of sending buy and sell orders to the electronic market, adjusting existing orders, deleting orders, or otherwise managing orders.

### BRIEF DESCRIPTION OF THE DRAWINGS

Example embodiments are described herein with reference to the following drawings, in which,

FIG. 1 is a block diagram illustrating an example electronic trading system in which a trader or user terminal and a gateway device are on a local area network;

FIG. 2 is a combination of a comparative shopping window with trading capabilities

FIG. 3 illustrates the pairing of an electronic trading system and a health care compliance management

FIG. 3.1 illustrates a rules-based system for mitigating risk

FIG. 4 is a block diagram illustrating an example electronic trading system providing an overview of how price, order, and fill information are communicated to and from a remote trader or user terminal via a remote host;

FIG. 5 illustrates the flow of knowledge and rules, at query/data

FIG. 5.1 illustrating procurement of health care

FIG. 6A illustrates Prior Art FIG. 6 illustrates the benefits over prior art

### SUMMARY

According to the example embodiments described herein, the systems and methods are provided to mitigate the risks of excessive charges in an electronic trading environment. To illustrate the present invention and aspects thereof, the following description, including the figures and detailed description, provides examples that can be used or readily modified by one of ordinary skill to generate a system or method that benefits from the teaching described and claimed herein.

In a remote host trading environment where a trader or user terminal communicates with a gateway via a remote host, a connection proxy may be located at the remote host. According to an example embodiment when connection is being established between the trader or user terminal and the gateway to enable a trader or user to trade at an electronic exchange, the connection stores product table, login information, subscription data as well as other data download that are provided to the trader or user terminal at the initialization stage.

Other features of the present invention will become more apparent to persons having ordinary skill in the art to which the present invention pertains from the following description and claims.

### DETAILED DESCRIPTION

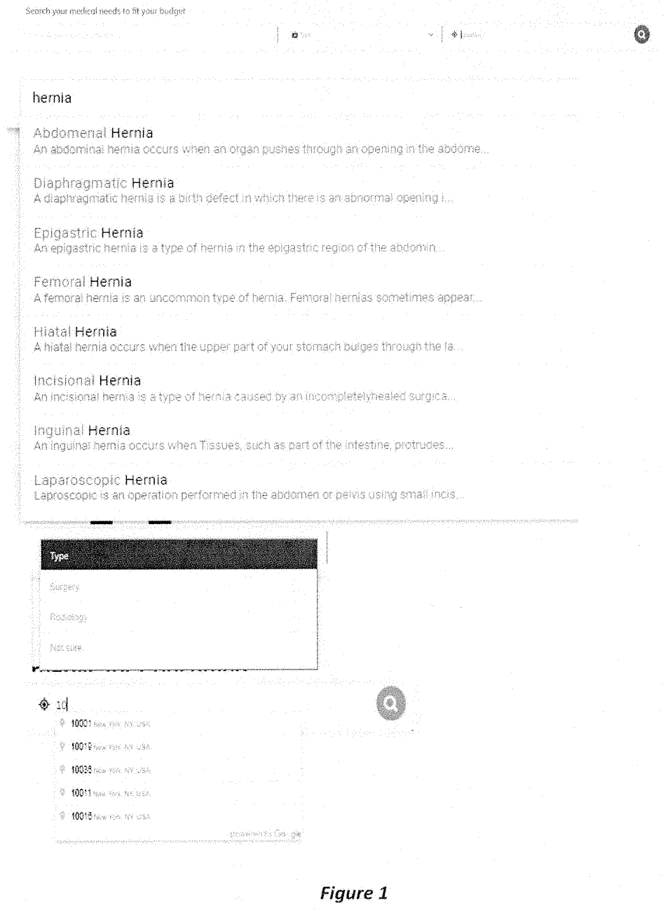

FIG. 1 illustrates a search box. The Search Box is on the Toolbar and is accessible from most of the screens. It is used to search terms to quickly find the code. The user can click the link “Help with Search? next to the Search button to see this information about how best to utilize the Search Box.

The system can search for any word in size of 2 characters up to 22 characters in length. To search for terms:

Type up t four key words for a term search, or a code number in the search box.

Click the Search button or press the Enter key to initiate a search.

For term searches, the user can specify a code set from the drop-down list, the program searches the specific code set for codes that relate to the terms entered. The program searches all code set databases for a specific match based on the entry. The Code set drop down is not applicable to numeric searches with specific codes.

Autofill allows the user to select a specific term. The autofill feature displays terms in alphabetical order but will only search the first term entered and provide resulting terms for the first term only. While autofill may not function beyond a single term, the user can still continue to type multiple terms and perform a search on the multiple terms typed into the search box.

1. Descriptions/Tabular

2. Lay Descriptions

3. Indexes

4. Clinical Classification Files

5. Terms to Code

6. Synonym Files

7. Coders Dictionary References

These sources will increase over time and new sources will be added to this list to increase both breadth and accuracy of the term search.

A minimum of three characters must be typed before auto complete begins. If you identify the term based on the autofill dropdown, you will be able to either mouse click on the term which will automatically execute the code search (without having to click the search button) OR you can use keyboard arrows to scroll down through terms and click the enter key on the highlighted term, which will execute the code search (as if the user clicked the search button).

When entering terms or codes to be searched, it is recommended to enter letters (A-Z) and/or numbers (0-9), and limited symbols such as basic punctuation (period, comma), avoid the use of other symbols, as these may cause search result errors.

The results of the autofill will return in A-Z order.

When entering ICD-9-CM or ICD-10-CM codes, it is possible to enter a code that can pertain to both ICD-9 and ICD-10 codes. When this is the case, the system will display the code in the User Defined Code List screen as it pertains to the ICD-9 code set and also as it pertains to the ICD-10 code set. These are called overlap codes and mostly occur with the E code series and the V code series.

ICD-10-PCS codes can be searched by entering the code up to the 4th character or the full 7th character.

If the search is too broad and has the potential to have too many results, a message will be displayed. You can narrow your search and limit the results by entering more information within the search box before executing the search process.

You can also type in a valid code number to bypass the term search and go directly to the Code Section or Code Detail for that code. The Code Set drop down box is ‘ignored’ in this situation.

You can type multiple code numbers separated by commas in the Search Box.

If you do not enter anything in the Search box and click Search, or press the Enter key, you will receive a Search Term Error message that indicates that you must enter search information before a search can be initiated. Remember, if you type a numeric code in the search field then this drop down is not applicable.

### Determining Search Criteria

When choosing search criteria, it is important to use terms that best identify the service or symptom. Entering the proper terms ensures that searching is quick and accurate. The suggestions below help to determine which terms to enter as search criteria.

The system can search for any word in size of 2 characters up to 22 characters in length. Select the Code Set Search radio button and specify a code set from the drop-down list to search the specific code set.

### Criteria for Procedure Codes

To determine the criteria for a procedure code search, the system determines what is being purchased., and where (anatomical site). For example, the key terms “arthroscopy shoulder” answers both what (arthroscopy) and where (shoulder). Entering these two terms brings up all codes containing arthroscopy and shoulder.

To further narrow the search, type one or two more terms that further explain the procedure. For example, entering “arthroscopy shoulder decompression” narrows the results to just one CPT Code, 29826, which contains all three terms. Avoid being too specific or entering too many search terms. In these instances, there may not be results that exactly match your criteria. It is best to m two to four key search terms.

### Criteria for Diagnosis Codes

To identify the criteria for an CM code search, using a crosswalk table, the system links the symptoms, conditions, diseases, to the matching procedural codes. The system will match up to four key terms that best describe the reason for seeking health care. For example, if a shoulder arthroscopy is being purchased, the system will link the most likely reasons for seeking that health care. Entering “joint tear” returns a large variety of matches in the Tabular Results. To return more specific results, the user is prompted to include the specific anatomical region. Entering “joint tear shoulder” returns only the diagnosis codes specific to the shoulder.

To identify the criteria for a Vol. 3 code search, the system determines what kind of procedure is being purchased, (e.g., appendectomy, hysterectomy), and enter this term into the Search Prompt. To further define y search, include the anatomical site where the procedure will be performed (e.g., abdominal region, pelvis).

### Spell Checking Search Terms

When you enter a search term that is either spelled incorrectly or not found in the code databases, the Alternative Spelling screen appears. Alternative search terms are listed for the search term you originally entered and are based on the first letter of that term or a phonetic match. This screen will show up to 100 alternative spellings organized in 4 columns. If there are more than 100 alternative spellings, you can click the ‘next page’ option at the top or bottom of the screen to display then next page of alternatives.

To select an alternative search term from the spell check screen:

1. Select the desired search term suggestion by clicking that term's radio button.

2. Click Search, which corrects your search term and closes the Spell Check dialog box. Doing this automatically continues your search based on the alternative term.

3. Click Cancel if you want to cancel out of this dialog box.
If you enter more than one search term, click the radio button next to all terms before clicking Search. Clicking IGNORE THIS TERM on any of the misspelled words and then clicking Search ignores that term but continues your search on all remaining words in your original search.

### Search Results

After you initiate a term search, the Search Results screen displays the matches found in all applicable code sets. You can set your desired code sets in the Code Sets Drop-down list on the Toolbar.

If there are too many results for the criteria entered a message will be displayed. You can limit the possible results by entering up to 4 terms in the search box.

The Search Results screen also includes the subsection range that the code belongs to.

### Viewing Additional Digit ICD Codes

The Search Results screen displays the code results. The parent grouping for ICD codes is the category code number without a decimal. ICD codes with additional digits are consolidated into a common category. Codes that require additional digits have a plus sign to the left of their folder.

Click the plus to expand the category and display subcodes.

Click the minus sign to hide subcodes.

### Viewing CPT and HCPCS Codes

CPT and HCPCS codes are consolidated into subsections identified by the range of codes in that subsection. For example, if CPT code 21031 Excision of torus mandibularis is part of your results, it is listed under this subsection range: 21015-21070 Musculoskeletal System, Head, Excision. Each subsection range has a plus sign to the left. Click the plus to expand the subsection listing and display codes.

When you have narrowed your search results sufficiently, you can click any code in the Search Results list to select it. The full description for the code and all neighboring codes is displayed on the right side of your screen in the CPT Code Detail or the HCPCS Code Detail.

To go to the code in the Code Detail, click any code from the Code Sections.

After you initiate a search, the Search Results screen displays the matches found in the applicable code set.

The Search Results screen also includes the subsection range that the code belongs to.

### Viewing Additional Digit ICD Codes

The Search Results screen displays the code results. The parent grouping for ICD codes is the category code number without a decimal. ICD codes with additional digits are consolidated into a common category. Codes that require additional digits have a plus sign to the left of their folder.

Click the plus to expand the category and display subcodes.
Click the minus sign to hide subcodes.

CPT® and HCPCS codes are consolidated into subsections identified by the range of codes in that subsection. For example, if CPT code 21031 Excision of torus mandibularis is part of your results, it is listed under this subsection range: 21015-21070 Musculoskeletal System, Head, Excision. Each subsection range has a plus sign to the left. Click the plus to expand the subsection listing and display codes.

When you have narrowed your search results sufficiently, you click any code in the Results to select it. The full description for the code and all neighboring codes is displayed on the right side of your screen in the CPT Code Detail or the HCPCS Code Detail.

### ICD-10-PCS Search Results

To access the Search Results screen, perform a code search or enter search terms. From here, click on a code to go to the ICD-10-PCS Code Section.

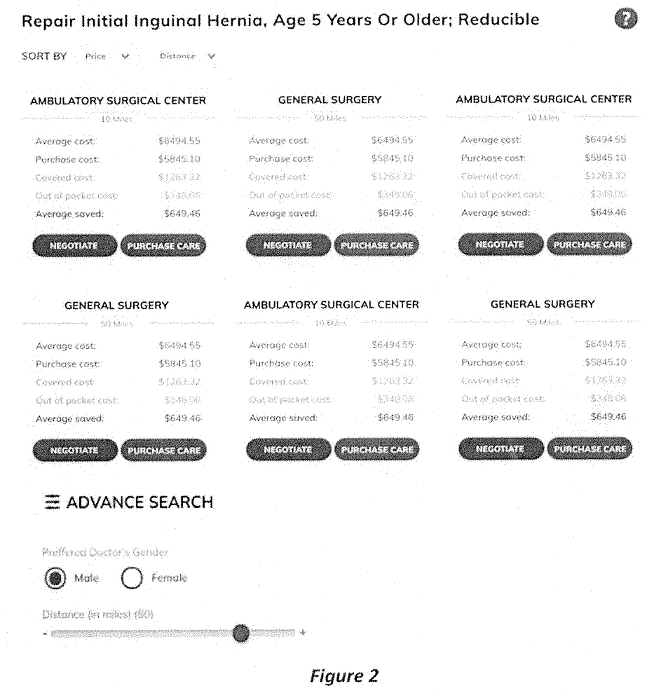

FIG. 2 illustrates a comparative shopping window. The user can click on the Search button or press Enter key to initiate a search. After performing a code search the system maps the designated code to provider registrants, matching one or more code sets to providers and their negotiated wholesale prices. The system will map the location on FIG. 1 zip code and pull all providers within a user defined radius. When a provider is matched to the user's selected code sets, a price matrix will link the provider ask price to the provider's zip code, specialty, prevailing charges. The user can expand the radius bar to shop for lower health care costs.

In the context of FIG. 1, a hernia search was initiated. At the client sever level, hernia is matched to hernia type.

The user selected inguinal hernia from the search box drop down. At the client sever level, inguinal hernia repair is automatically coded to CPT 49505. CPT code 49505 is linked to providers with experience in performing inguinal hernia surgery. FIG. 2 displays a comparative shopping window with the option of directly bidding or purchasing the inguinal surgical hernia repair from within the comparative shopping window. If the bid is too low, the system will alert the user that the bid is below market prices.

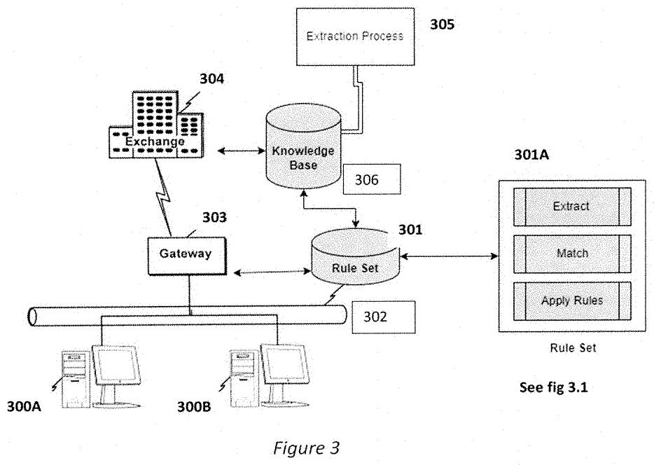

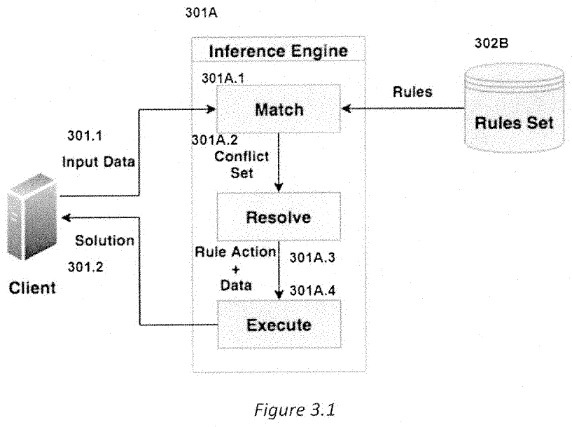

FIG. 3 illustrates a knowledgebase 305, a data extraction process.

In one embodiment the knowledgebase gathers external data and proceeding to extract the structured and unstructured data for the purpose of gaining knowledge.

Structured and unstructured data is transformed to knowledge, knowledge built on pattern matching; which includes, but is not limited to pairing medical condition to medical code sets; pairing health care costs to where the health care costs are generated, i.e., a free-standing surgical facility compared to a hospital outpatient facility; pairing an episode of care to a continuum of care, to pairing code sets to provider scope of license, to pairing code sets to duration of care; pairing adjudication rules from one insurance company to another; pairing facts and data against the set of rules.

Further comprising of matching facts and data against the set of rules. Extracting rules and data to extract new rules. Further comprising of the use of an inference engine, which is a component of the knowledgebase, that applies logical rules to the knowledgebase to infer new information from known fact, i.e., derived from the pairing of health care data.

The knowledgebase captures and uses knowledge from a variety of health care related sources. Source edits include but are not limited to edits and rules from the American Medical Association (AMA) CPT guidelines, CMS guidelines, Correct Coding initiatives, commercial and Medicare code sets, medical societies, and other widely accepted third-party sources.

The knowledgebase includes a comprehensive set of rules for commercial and government sponsored edits that map to place of service edits, like facilities i.e., hospitals and stand-alone surgical centers. The knowledge base instantly reviews and edits any inpatient or outpatient health care services i.e., claims for errors, omissions, and questionable coding relationships by testing the data against an expansive knowledgebase containing millions of government and industry rules that cover health care claims.

The rules engine interface can instantly detect coding errors related to diagnosis-related group (DRG) and modifier appropriateness, mutually exclusive and incidental procedures, duplicate claims, revenue code and Healthcare Common Procedure Coding System (HCPCS) relationships, implantable device processing, and more.

The rules engine interface,

The mechanism instantly reviews medical necessity edits without supporting diagnoses codes or considered not medical necessary based on national coverage determination (NCD) and local coverage determination. Additional sourced edits and rules including but are not limited to HIPAA Code set rules; HIPAA Code Sets; any set of codes used for encoding data elements, such as tables of terms, medical concepts, medical diagnosis codes, or medical procedures codes;

In one embodiment, the knowledgebase has a three-tier filtering process,

1. Pre-processing: at this stage the processing is all about improving data being worked with and incorporating the concept of data cleaning, including data integration, heterogeneous data from multiple sources combined in a common source (data warehouse). Additionally, at this stage predictive models for unreliable data are established to predict similarly faulty, missing attributional mismatched data, then working it out in future processes.

2. This step concentrates on converting the pre-processed data to the fully utilizable trading and order data. Here the information is organized and sorted, often unified into a single type. For example, conditional orders are those which will only be executed or activated if certain criteria are met. Likewise, non-conditional orders are a normal instance in which orders placed have no specific price criteria.

3. Data processing, using data search capabilities and statistical algorithms to discover patterns and correlations, including anomalies in knowledgebase data.

4. Interpretation/Evaluation, this final phase is one during which the data is sent to a Rule Manager. The data has been cleaned, converted, picked apart based on relevant attributes.

The RBE is a component of the knowledgebase. The structured and the unstructured data samples are processed by computer, running samples of classes gathers data from structured and unstructured source through a classifier to train it to recognized or establish the elements that fall within a class, subsequently, a computer processor runs the trained classifier to map the code sets to the appropriate code sets. For instance, the code set range 10000-69990 are the elements that defined surgery; another example, code elements for radiology are 70000-79999. These elements can be used for performing statistical analysis of a code belonging to a class membership, i.e., inguinal hernia surgery is under the sub-type “hernia” falling within the category of surgery, which may include additional characteristics of hernia, such as inguinal hernia, which would be considered an anatomical site, i.e., groin, and the groin is bilateral.

In another embodiment, pattern matching, at this phase, the inference engine matches the facts and data against the set of rules.

That is, left groin or right groin, or both groins. These associated rules are joined to the code set class and a subset database can be used to pair these two conditions; where A=surgery and B=type of surgery is paired and mapped to the associated conditions database; to determine if C is matched to the database of associated rules. If C is matched, and C is confirmed by the buyer during the code search criteria phase to be true the adjudication of code set hernia, specifically code 49505, the RBE will reconsider its rational and match the rules and edits that are relevant to the bidding or purchase of a bilateral surgery event.

### EXAMPLE OF APPLYING A CLASSIFIER

1. Training: Training is an iterative process. The process involves taking content that is known content to belong to specified classes, i.e., CPT codes, and creating a classifier based on that known content. For example, training code set (10000-69999) is determined to belong to the class of Surgery.
Constitutive rules are extracted during the training process through data mining classification algorithms in such a manner that the training set is covered. The purpose of extracting decisions rules from training data is to obtain generalized rules that define classes of data. The generalization process ends when new rules cannot be generated, or the specified termination criteria are met. The extraction of constitutive rules is used to classify the samples in the test set within the knowledgebase data.

2. Example of a classification step, Applying the Surgery classifier training content (10000-69999) to match a buyer search criteria input from the search box and determining the class membership of the Buyer's designated code.

In one embodiment, an inference engines manages many rules and facts inside the electronic exchange. Inside the electronic exchange, a Rule Manager is picking rules and applying on data and generating solutions during the extraction process.

The Rule Manager runs on the trading data and if any condition matches then it executes the corresponding actions. The following sampling of data Conditions include but are not limited to the other pickings of the Rule Manager.

### EXAMPLES OF EXTRACTED DATA CONDITIONS

Category: Standard preparation/monitoring services for anesthesia:
Condition: Reason for price reduction; The physician determines which preparation and monitoring services are utilized for an anesthesia procedure. These services are included in the anesthesia service. Accordingly, when reporting the anesthesia service code,
Binary attributes: Accordingly, when reporting the anesthesia service code, HCPCS/CPT code______(the column one HCPCS/CPT code), the services described by HCPCS/CPT code______(the column two HCPCS/CPT code) are included in the anesthesia service. Target price for column 2 is zero
Value proposition benefits: A reduction in health care cost.
Category: HCPCS/CPT procedure code definition:
Condition: If a HCPCS/CPT code is reported along with other HCPCS/CPT codes that are components of the descriptor of the first code, only the first code should trade.
Binary attributes: The HCPCS/CPT code______(the column one HCPCS/CPT code) descriptor includes the service described by the descriptor of HCPCS/CPT code______(the column two HCPCS/CPT code). Thus, based upon the HCPCS/CPT code descriptors, HCPCS/CPT code______(the column two HCPCS/CPT code) is bundled into HCPCS/CPT code______(the column one HCPCS/CPT code), and column two code is adjusted to a price target value of 0$ under this condition.
Value proposition: A reduction in health care cost.
Category: CPT Manual or CMS manual coding instruction:
Condition: In addition to CPT procedure code definitions or descriptors or CMS issues coding instructions and guidelines in various database related publications;
Binary attributes: In the case of HCPCS/CPT code______(the column one HCPCS/CPT code) and HCPCS/CPT code_____(the column two HCPCS/CPT code), CPT or CMS instructions identify appropriate methodology for code submission and accordingly, HCPCS/CPT code______(the column two HCPCS/CPT code) is included in or cannot be purchased with HCPCS/CPT code______(the column one HCPCS/CPT code), and column two code is adjusted to a price target value of 0$ under this condition.
Value proposition: A reduction in health care cost.
Category: Mutually exclusive procedures:
Condition: certain services or procedures are listed which would not reasonably be performed at the same session by the same provider on the same beneficiary.
Binary attributes: In the case of HCPCS/CPT code______(the column one HCPCS/CPT code) and

HCPCS/CPT code______(the column two HCPCS/CPT code), it would be unreasonable to expect these services to be performed at a single patient encounter and, therefore, these HCPCS/CPT codes have been paired together as edits. Column two HCPCS/CPT order/code should not be purchased; and column two code is adjusted to a price target value of 0$ under this condition.

### 1. A First Example Trading Mechanism

FIG. 3 illustrates an example electronic trading system 300 in which the example embodiments may be employed. The example system 300 comprises trading stations 300A and 300B that access an electronic exchange 304 through a gateway 303 As illustrated in FIG. 1, the trading stations 300A-B and the gateway 303 are located within a local LAN 302, although other configurations are possible. While not shown, a router could be used to route messages between the gateway 303 and the electronic exchange 304. The electronic exchange 304 includes a computer process (e.g., the central computer) that matches buy and sell orders sent from the trading stations 300A-B with orders from other trading stations (not shown). The electronic exchange 304 may list one or more tradeable objects for trading. While not shown in FIG. 1 for the sake of clarity, the trading system may include other devices that are specific to the client site like middleware and security measures like firewalls, hubs, security managers, and so on, as understood by persons skilled in the art. FIG. 1 illustrates an example electronic trading system 300 in which the example embodiments may be employed. The example system 300 comprises trading stations 308A and 308 B that access an electronic exchange 304 through a gateway 303. As illustrated in FIG. 1, the trading stations 300-B and the gateway 303 are located within a local LAN 302, although other configurations are possible. While not shown, a router could be used to route messages between the gateway 303 and the electronic exchange 304 The electronic exchange 304 includes a computer process (e.g., the central computer) that matches buy and sell orders sent from the trading stations 300A-B with orders from other trading stations (not shown). The electronic exchange 304 may list one or more tradeable objects for trading. While not shown in FIG. 1 for the sake of clarity, the trading system may include other devices that are specific to the client site like middleware and security measures like firewalls, hubs, security managers, and so on, as understood by persons skilled in the art.

Regardless of the types of order execution algorithms used, the electronic exchange 304 provides market information to the subscribing trading stations 300A-B. Market information may include data that represents just the inside market. The inside market is the lowest sell price (best ask) and the highest buy price (best bid) at a particular point in time. Market information may also include market depth. Additionally, the exchange 304 can offer other types of market information such as the last traded price (LTP), the last traded quantity (LTQ), and order fill information.

The computer employed as the trading stations 300A-B generally can range from a hand-held device, laptop, or personal computer to a larger computer such as a workstation and multiprocessor. An illustrative personal computer may use Pentium™ microprocessors and may operate under a Windows operating system, or yet may use some other microprocessor or operating system. Generally, the trading stations 300A-B includes a monitor (or any other output device) and an input device, such as a keyboard and/or a two or three-button mouse to support click based trading, if so desired. One skilled in the art of computer systems will understand that the present example embodiments are not limited to any particular class or model of computer employed for the trading stations 300A-B and will be able to select an appropriate system.

In one example embodiment, the trading stations 300A-B use software to create specialized interactive trading screens on terminals associated with them. Trading screens preferably enable traders to, among other things, enter and execute orders, obtain market quotes, and monitor positions. The range and quality of features available to the trader on his or her trading screen may vary according to the specific software application being run. In addition to or in place of the interactive trading screens, a trading station could run automated types of trading applications.

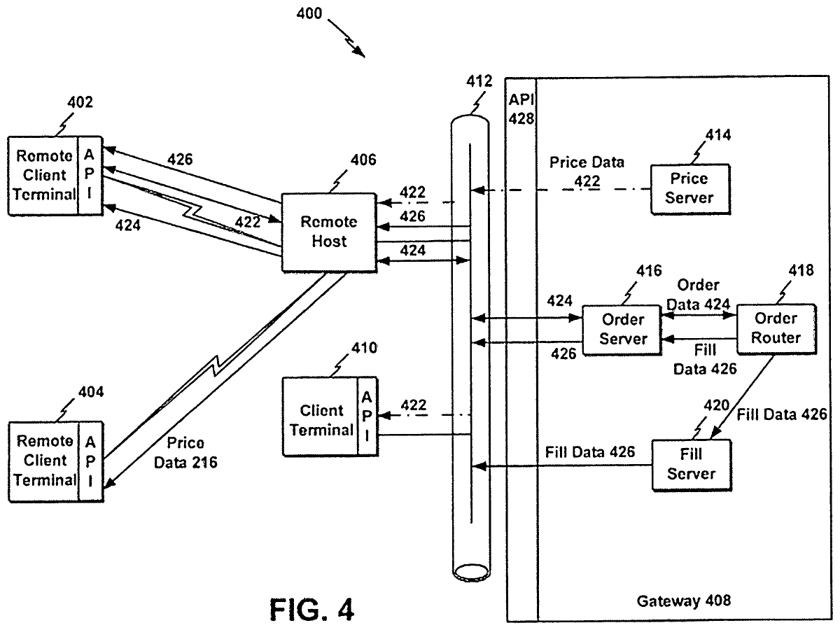

It should be noted that orders in the system illustrated in FIGS. 1 and FIG. 4 could also be placed using any other trading application as well. Additionally, the preferred embodiments are not limited to any particular product that performs translation, storage, and display function.

The computer employed as the gateway 303 generally can range from a personal computer to a larger or faster computer. An illustrative gateway computer may use Pentium™ microprocessors and may operate under a Windows (server or workstation) operating system, or yet some other system. Generally, the gateway 303 may additionally include a monitor (or any other output device), input device, and access to a database, if so desired. One skilled in the art of computer systems will also understand that the present example embodiments are not limited to any particular class or model of computer(s) employed for the gateway 303 and will be able to select an appropriate system. Additionally, in some instances, a gateway, such as gateway 303, may not even be necessary and/or another type of network intermediary device may be employed.

It should be noted that a computer system that may be employed here as a trading station or a gateway generally includes a central processing unit, a memory (a primary and/or secondary memory unit), an input interface for receiving data from a communications network, an input interface for receiving input signals from one or more input devices (for example, a keyboard, mouse, etc.), and an output interface for communications with an output device (for example, a monitor). A system bus or an equivalent system may provide communications between these various elements.

Memory on either the gateway 303 or the trading station 300 A-B may include a computer readable medium. The term computer readable medium, as used herein, refers to any medium that participates in providing instructions to a processor unit for execution. Such a medium may take many forms, including but not limited to, non-volatile media, and transmission media. Non-volatile media include, for example, optical or magnetic disks, such as storage devices. Volatile media include, for example, dynamic memory, such as main memory or random-access memory (“RAM”). Common forms of computer readable media include, for example, floppy disks, flexible disks, hard disks, magnetic tape, punch cards, CD-ROM, any other physical medium, memory chip or cartridge, or any other medium from which a computer can read.

It should also be noted that the trading stations 300A-B generally execute application programs resident at the trading stations 300A-B under the control of the operating system of the trading station. Also, the gateway 303 executes application programs resident at the gateway 303 under the control of the operating system of the gateway 303. In other embodiments and as understood by a person skilled in the art, the function of the application programs at the trading stations 300A-B may be performed by the gateway 303, and likewise, the function of the application programs at the gateway 303 may be performed by the trading stations 300A-B.

The actual electronic trading system configurations are numerous, and a person skilled in the art of electronic trading systems would be able to construct a suitable network configuration. For the purposes of illustration, some example configurations are provided to illustrate where the elements may be physically located and how they might be connected to form an electronic trading system. These illustrations are meant to be helpful to the reader, and they are not meant to be limiting. According to one example, the gateway device may be located at the client site along with the trading station, which is usually remote from the matching process at the electronic exchange. According to another example, the gateway device may be located at the exchange side. As such, the present invention is not limited to any actual network configuration.

According to the illustrated embodiment, the trading stations 300A-B, the gateway 303, and any routers communicate over the LAN 302, and the gateway 303 may communicate with the matching process at the electronic exchange 304 over a T1, T3, ISDN, or some other high-speed connection. In another example illustration, the client site may be located on the actual grounds of the electronic exchange (for example, in the building of the exchange). According to this instance, the trading station and the gateway may still communicate over a LAN, but if any routers are used, they may communicate with the matching process at the electronic exchange through another connection means besides a T1, T3, or ISDN. In yet another example illustration, the gateway may be housed at, or near, its corresponding electronic exchange. According to this instance, the trading station may communicate with the gateway over a wide area network or through the use of a T1, T3, ISDN, or some other high-speed connection.

While a single exchange is shown in FIG. 1, it is understood that a trader may obtain access and trade at multiple electronic exchanges. In such an embodiment, a client terminal could access multiple exchanges through multiple gateways, with each gateway designated for a specific exchange. Alternatively, a single gateway may be programmed to handle more than one electronic exchange.

It could be very valuable to provide a trader with the opportunity to trade tradeable objects listed at different electronic exchanges. For example, a trader could view market information from each tradeable object through one common visual display. As such, price, and quantity information from the two separate exchanges may be presented together so that the trader can view both markets simultaneously in the same window. In another example, a trader can spread-trade different tradeable objects listed at the different electronic exchanges. Regardless, the present invention is not so limited.

FIG. 3 illustrates the pairing of trading with an automated compliance manager. FIG. 3 further illustrates an integrated system of processes, tools, controls, and function to manage risk. An inference engine manages a large number of rules and facts inside the health care expert system. The virtual compliance manager runs the rules on the financial transaction and if any condition matches then it executes the corresponding action.

FIGS. 3 and 3.1 illustrate a rule set 301, a pattern matching process: the rule set sends data to the inference engine. The inference engine matches the facts and data against the set of rules. In a further embodiment, the inference engine manages the order of conflicting rules, wherein resolving the conflict, and giving the selected one rules. The inference engine simply runs the action of the selected rule on any given data associated with a financial transaction and returns the output/result to the trader/user.

FIG. 4 A Second Example Trading Mechanism

FIG. 4 is an example of an electronic trading system 400 in which the example embodiments may be employed.

FIG. 4 is a block diagram of a system 400 illustrating an example method for providing and receiving price, order and fill information to and from a client terminal and a gateway in a remote host electronic trading environment. The system 400 includes two remote client terminals 402 and 404, a remote host 406, and a gateway 408. Just to give an example of a system that combines both local and remote communication systems, the system 400 illustrates a local trading station 410 that communicates with the gateway 408 via a LAN 412.

Rather than creating API connection between the gateway 428 and the remote client terminals, API connections are created to and from the remote host 406 that communicates data to the remote client terminals 402 and 404.

The gateway 408 includes a price server 414, an order server 416, an order router 418, and a fill server 420. The gateway 408 could include additional components as well. In a remote connection trading environment, the price server 414 sends price data 422 to the remote host 406 via multicast using a multicast address. The remote host 406 is configured to listen to the multicast and receive the price data 422 just like any other subscribing trading station located on the LAN, such as the trading station 410. Then, the remote host 406 forwards the received price data to each subscribing remote client terminal, such as the terminals 402 and 404, via a separate TCP connection created for each client terminal.

In a trading environment using a remote connection to a trading network, orders are submitted at the remote client terminals 402 and 404. The client terminals 402 and 404 may then create order messages and send the order messages to the remote host 406 vii a TCP connection, such as shown at 424 with respect to client terminal 402. Upon receiving an order message, the remote host 406 forwards the order message to the order server 416 using a unicast connection. At this point, the order information is passing through the local LAN. Once the order server 416 receives the order message, it sends the order message to an electronic exchange. Also, the order server 216 sends an order confirmation back to the remote host 406, and the host 406 then forwards the confirmation to the remote client terminal 402 (or 404) via the TCP connection. When a fill is sent to the gateway 408 in a remote electronic trading environment, the fill confirmation is created at the fill server 420 and the order server 416, and it is sent from the gateway 408 to the remote host 406 as shown at 426. Once the fill confirmation is received at the remote host 406, the host 406 forwards it to the remote client terminal 402 via a TCP connection. While not specifically described, it should be understood that the remote host 406 may repackage any messages that are received from and sent to the remote client terminals 402 and 404. Similarly, the servers at the gateway 408 may process and change format of messages or other data that are received from and sent to an electronic exchange and client terminals.

While two example trading systems have been described above, it should be understood that different network connections are possible, and the present invention is not limited to the described example systems. For example, a trading system could be configured to use Wide Area Networks (WAN) and WAN routers or wireless networks. One skilled in the art of electronic trading systems will understand that the present embodiments are not limited to the particular configurations illustrated and described with respect to FIG. 1 and FIG. 4 and will be able to design a particular system based on the specific requirements (for example, by adding additional exchanges, gateways, trading stations, routers, or other computers serving various functions like message handling and security).

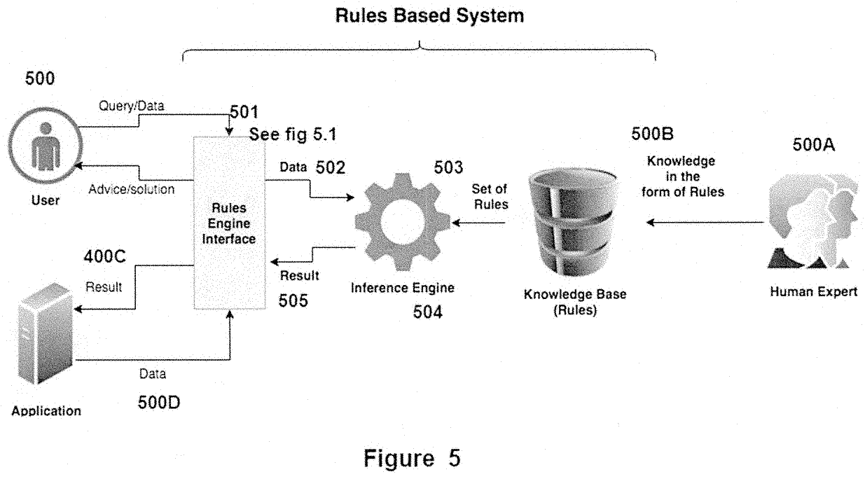

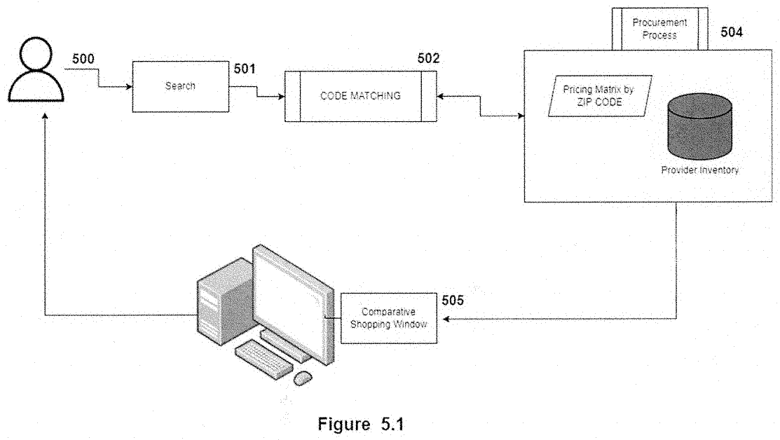

FIG. 5 illustrates the development of rules 505 and the application of the rules at 500. The following represents examples extracted from the process illustrated in FIG. 5:

### EXAMPLES OF EXTRACTED DATA CONDITIONS

Category: Standard preparation/monitoring services for anesthesia:
Condition: Reason for price reduction; The physician determines which preparation and monitoring services are utilized for an anesthesia procedure. These services are included in the anesthesia service. Accordingly, when reporting the anesthesia service code,
Binary attributes: Accordingly, when reporting the anesthesia service code, HCPCS/CPT code______(the column one HCPCS/CPT code), the services described by HCPCS/CPT code______(the column two HCPCS/CPT code) are included in the anesthesia service. Target price for column 2 is zero
Value proposition benefits: A reduction in health care cost.
Category: HCPCS/CPT procedure code definition:
Condition: If a HCPCS/CPT code is reported along with other HCPCS/CPT codes that are components of the descriptor of the first code, only the first code should trade.
Binary attributes: The HCPCS/CPT code______(the column one HCPCS/CPT code) descriptor includes the service described by the descriptor of HCPCS/CPT code______(the column two HCPCS/CPT code). Thus, based upon the HCPCS/CPT code descriptors, HCPCS/CPT code______(the column two HCPCS/CPT code) is bundled into HCPCS/CPT code (the column one HCPCS/CPT code), and column two code is adjusted to a price target value of 0$ under this condition.
Value proposition: A reduction in health care cost.
Category: CPT Manual or CMS manual coding instruction:
Condition: In addition to CPT procedure code definitions or descriptors or CMS issues coding instructions and guidelines in various database related publications;
Binary attributes: In the case of HCPCS/CPT code______(the column one HCPCS/CPT code) and HCPCS/CPT code______(the column two HCPCS/CPT code), CPT or CMS instructions identify appropriate methodology for code submission and accordingly, HCPCS/CPT code______(the column two HCPCS/CPT code) is included in or cannot be purchased with HCPCS/CPT code______(the column one HCPCS/CPT code), and column two code is adjusted to a price target value of 0$ under this condition.
Value proposition: A reduction in health care cost.
Category: Mutually exclusive procedures:
Condition: certain services or procedures are listed which would not reasonably be performed at the same session by the same provider on the same beneficiary.
Binary attributes: In the case of HCPCS/CPT code______(the column one HCPCS/CPT code) and HCPCS/CPT code______(the column two HCPCS/CPT code), it would be unreasonable to expect these services to be performed at a single patient encounter and, therefore, these HCPCS/CPT codes have been paired together as edits. Column two HCPCS/CPT order/code should not be purchased; and column two code is adjusted to a price target value of 0$ under this condition.
FIGS. 2 and 5.1 illustrates the comparative shopping window. The following is an example of the bidding process:

1. From a comparative shopping window, the user can select providers based on price, quality of care, experience, by geographic location, i.e., zip code. The provider profile includes market data, including place of service, hospital affiliations;

2. Once the patient selects on a health care service or product, the user places an initial offer for the health care service or product.

3. If the price offered by the patient equals or is greater than the threshold price, the transaction takes place at the price quoted, (balance billing is eliminated)

4. If the offer is less than the threshold prices (the lowest price a service or product can be sold for) set by the Purchase Care Exchange (the seller), the patient gets a chance to update their offer in subsequent rounds.

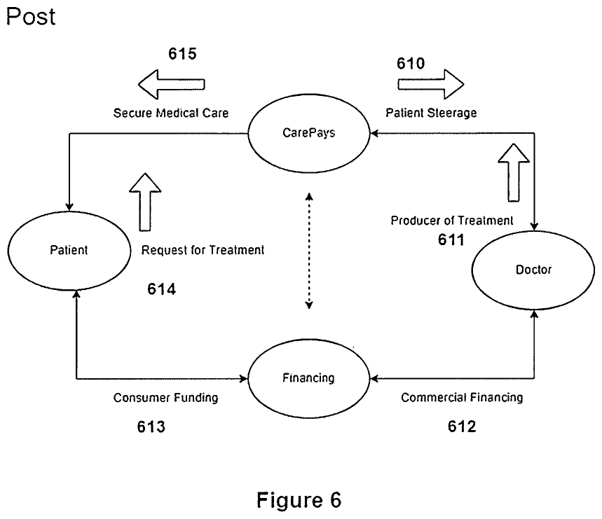

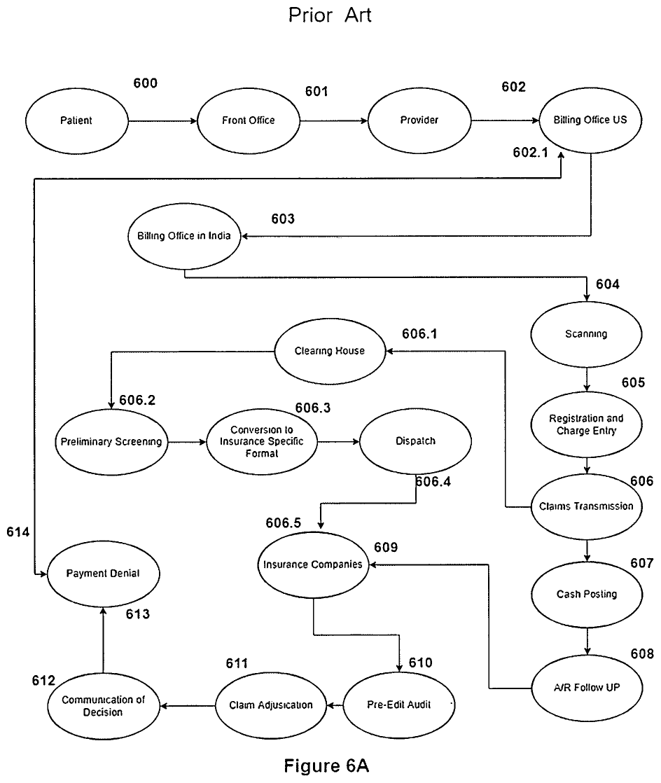

FIG. 6A illustrates prior art medical billing.

FIG. 6B illustrates the consolidation of efforts from prior art to a more efficient process.

to

What is claimed:

1. A non-transitory computer readable medium having stored therein instructions executable by a processor, wherein the instructions are executable to:
provide by a connection proxy a signal to an intermediary device, wherein the signal is provided in response to detecting that a network communication link between a client terminal and the intermediary device has been established, wherein the client terminal communicates with an electronic exchange using the communication session established for the client terminal at the intermediary device;
received by the connection proxy market data related to a tradeable health care; and
provide by the connection proxy initial download data and the market data related to the tradeable health care to the client terminal.

2. The computer readable medium of claim 1, wherein the connection proxy is located at the intermediary device.

3. The computer readable medium of claim 1, wherein the intermediary device includes a gateway.

4. The computer readable medium of claim 1, wherein the instructions to provide by the connection proxy the signal to the intermediary device to maintain the communication session at the intermediary device include instructions further executable to:

5. The computer readable medium of claim 1, wherein knowledge is stored in the form of rules (if-then form).

6. The computer readable medium of claim 1, wherein the intermediary device runs the rules on the data and if any condition matches then it executes the corresponding actions.

7. The computer readable medium of claim 1, wherein the market data includes price data.

8. The computer readabl8 medium of claim 1, wherein the market data includes market depth data.

9. The computer readable medium of claim 1, wherein the market data includes order fill data.

10. The computer readable medium of claim 1, wherein the market data includes order confirmation data.

11. The computer readable medium of claim 1, wherein the instructions are further executable to:
receive by the communication proxy the initial download data; and
store by connection proxy the initial download data.

12. The computer readable medium of claim 1, wherein the initial download data includes initialization data.

13. The computer readable medium of claim 1, wherein the initial download data includes subscription data.

14. The computer readable medium of claim 1, wherein the initial download data includes data related to at least one tradeable health care code set being traded at the electronic exchange.

15. The computer readable medium of claim 1, wherein the instructions are further executable to: update by the connection proxy the initial download data during a trading day.

16. A computer readable medium having stored herein instructions executable by a processor, wherein the instructions are executable to:
Receive by the connection proxy market related to a tradable health care
Provide by the connection proxy initial download data and market data related to the tradable health care to the client terminal to the client terminal and the intermediary device; [symbol]
